(19) United States
(12) Patent Application Publication
(10) Pub. No.: US 2024/0203571 A1
(43) Pub. Date: Jun. 20, 2024

(54) SYSTEMS AND TECHNIQUES FOR MANAGING CONTROLLED SUBSTANCE INVENTORY DATA

(71) Applicant: VETSNAP CORP., Thousand Oaks, CA (US)

(72) Inventors: Steven Patrick ELLIOTT, Thousand Oaks, CA (US); Yangyang SHI, Thousand Oaks, CA (US)

(21) Appl. No.: 18/380,046

(22) Filed: Oct. 13, 2023

Related U.S. Application Data
(60) Provisional application No. 63/433,349, filed on Dec. 16, 2022.

(51) Int. Cl. G16H 40/20; G16H 20/13

(52) U.S. Cl. CPC G16H 40/20; G16H 20/13

(57) ABSTRACT

Systems and techniques for managing a controlled substance inventory of a medical practice are disclosed. A platform application enabling entry of inventory and usage records of one or more controlled substances for the medical practice is provided. The platform application receives, from a computing device, a request to record entry of a controlled substance into a digital log book associated with the medical practice. The platform application generates a virtual container to associate with the controlled substance and associates the virtual container to the digital log book.

### CROSS-REFERENCE TO RELATED APPLICATIONS

This patent application claims priority to U.S. Provisional Patent Application Ser. No. 63/433,349, filed on Dec. 16, 2022, entitled “SYSTEM AND TECHNIQUES FOR MANAGING CONTROLLED SUBSTANCE INVENTORY DATA,” which is herein incorporated by reference in its entirety.

### FIELD

Embodiments disclosed herein generally relate to practice management and inventory systems, and more specifically, to managing controlled substance inventory using such systems.

### BACKGROUND

In the veterinary industry, a medical practice (e.g., a clinic, hospital, pharmacy, etc.) must, by law, record and track usage of controlled substances in patients. Generally, controlled substances are chemicals, pharmaceutical agents, and the like that have been identified by a governmental body as having a potential for abuse. For example, the United States Drug Enforcement Agency (DEA), pursuant to the Controlled Substances Act (CSA), categorizes and regulates controlled substances based on medicinal use, potential for abuse, and safety or dependence liability.

Tracking usage of controlled substances typically involves two sources: a controlled substance log and a practice information management system (PIMS). An individual at the practice (e.g., a veterinary doctor, assistant, or other staff) must input usage information in the controlled substance log as the usage occurs throughout the day. The PIMS maintains patient medical records, which includes itemized controlled drug substance usage for that patient entered generally for accounting purposes.

The controlled substance log is subject to audits to ensure that the practice is accurately tracking usage. During an accountability audit of a medical practice, such as a veterinary clinic, a DEA investigator conducts an on-site inspection of the clinic to verify, among other things, that the clinic is accurately accounting for controlled substances being managed and administered. For instance, the investigator may, through the audit, determine whether the clinic has maintained complete and accurate records and effective security. To this end, the clinic furnishes an audit report, which discloses any variance in the quantity of controlled substances accounted for and what it the practice is responsible to account for during a given time period. The audit report is subject to a number of requirements and attempts to balance the controlled substances on-hand at the beginning of the period plus receipts against dispositions plus quantities on-hand at the end of the period.

Because typical inventory management systems of a medical practice often use a number of sources, such as a controlled substance log and a PIMS, preparing the audit report itself can be time-consuming. For instance, the process typically requires reconciliation of inventory and usage data maintained in a physical or digital controlled substance log and the PIMS, which can be error prone and inefficient. Consequently, to maintain compliance with controlled substance regulations, the audit reports must provide a complete and accurate accounting for the controlled substances on a day-to-day basis. However, current approaches for doing so are capable of capturing an incomplete snapshot of the accounting and can also rely on manual checks (typically error prone and inefficient) for missing entries and unmatched levels of usage.

### SUMMARY

Embodiments presented herein disclose systems and computer-implemented methods for managing controlled substance inventory and usage data of a medical practice. One embodiment of the present disclosure generally provides a system having one or more processors and a memory. The memory stores instructions, which, when executed on the one or more processors, causes the system to provide a platform application enabling, from computing devices, entry of inventory and usage records of one or more controlled substances for a medical practice. The system receives, via the platform application from a first computing device, a request to record entry of a first controlled substance into a digital log book associated with the medical practice. The request specifies at least a date of the entry and a quantity of the first controlled substance. The system generates, via the platform application, a virtual container including the specified information to associate with the first controlled substance and associates the virtual container to the digital log book.

Another embodiment of the present disclosure provides a method. The method generally includes providing a platform application enabling, from computing devices, entry of inventory and usage records of one or more controlled substances for a medical practice. The method also generally includes receiving, via the platform application from a first computing device, a request to record entry of a first controlled substance into a digital log book associated with the medical practice. The request specifies at least a date of the entry and a quantity of the first controlled substance. A virtual container to associate with the first controlled substance is generated. The virtual container includes the specified information. The virtual container is associated to the digital log book.

### BRIEF DESCRIPTION OF THE DRAWINGS

The concepts described herein are illustrated by way of example and not by way of limitation in the accompanying figures. For simplicity and clarity of illustration, elements illustrated in the figures are not necessarily drawn to scale. Where considered appropriate, reference labels have been repeated among the figures to indicate corresponding or analogous elements.

FIG. 1 illustrates a simplified conceptual diagram of an example embodiment computing environment in which controlled substance inventory and usage data from multiple sources is tracked and reconciled, according to an embodiment;

FIG. 2 is a simplified block diagram of an example embodiment of the platform server described relative to FIG. 1, according to an embodiment;

FIG. 3 is a simplified block diagram of an example embodiment of the client device described relative to FIG. 1, according to an embodiment;

FIG. 4 is a conceptual diagram illustrating virtual containers of controlled substances to be associated with a digital log book of a medical practice, according to an embodiment;

FIG. 5 is a simplified flow diagram of a method for entering a controlled substance into a digital log book, according to an embodiment;

FIG. 6 is a simplified flow diagram of a method for managing usage data for a controlled substance associated with a digital log book, according to an embodiment;

FIG. 7 is a simplified flow diagram of an example method for generating an accountability audit report of controlled substance inventory and usage for a medical practice, according to an embodiment; and

FIG. 8 is a simplified flow diagram of an example method for reconciling inconsistent quantity reporting for a digital log book, according to an embodiment.

### DETAILED DESCRIPTION

Embodiments presented herein disclose systems and techniques for managing controlled substance inventory and usage data. More particularly, embodiments provide a controlled substance data management platform that intakes, reconciles, and manages inventory and usage data from a number of sources that may be used by a medical practice, such as a controlled substance log and a practice inventory management system.

Further, these embodiments may be adapted to an inventory management system platform that integrates with a practice inventory management system (PIMS) of a medical practice, such as a veterinary practice. The platform may serve as a single and auditable source of controlled usage data, providing a controlled substance usage log that the veterinary practice can use (e.g., via a client device executing a platform application thereon) to enter standardized usage data throughout the day while treating patients while maintaining an accurate accounting by using virtual containers that are analogous to real-world equipment containers storing controlled substance at various quantities. The platform may also obtain, aggregate, discretize, and standardize data obtained from the PIMS to more efficiently validate data sets between the platform controlled substance usage log and the PIMS data.

For instance, to efficiently validate usage data between the controlled substance log of the platform and the PIMS, the platform architecture and PIMS may be arranged such that discrete functions of the validation process are carried out individually in distinct computer systems of the platform, such that computational resources of a single system can be allocated to a single function. Once validated, a resulting reconciled usage log can be generated and output to the platform application.

Advantageously, the platform, in integrating with each system involved with the inventory, management, and administration of controlled substances for a medical practice, maintains controlled substance inventory and usage data of a medical practice to a granular level not previously captured in preexisting systems. For example, the platform may manage controlled substance inventory through virtual containers, which are analogous to and may map to physical containers used to store a collection of controlled substances at a physical location. The platform may also keep records of movement to and from the virtual containers such that inventory throughout a given timeframe is trackable. As a result, the granular management and tracking of controlled substance inventory and usage creates a digital audit trail that the platform can reproduce in real-time in an audit report compliant with governmental regulatory agencies such as the United States Drug Enforcement Agency (DEA).

While the concepts of the present disclosure are susceptible to various modifications and alternative forms, specific embodiments thereof have been shown by way of example in the drawings and will be described herein in detail. It should be understood, however, that there is no intent to limit the concepts of the present disclosure to the particular forms disclosed, but on the contrary, the intention is to cover all modifications, equivalents, and alternatives consistent with the present disclosure and the appended claims.

References in the specification to “one embodiment,” “an embodiment,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described. Additionally, it should be appreciated that items included in a list in the form of “at least one A, B, and C” can mean (A); (B); (C); (A and B); (A and C); (B and C); or (A, B, and C). Similarly, items listed in the form of “at least one of A, B, or C” can mean (A); (B); (C); (A and B); (A and C); (B and C); or (A, B, and C).

The disclosed embodiments may be implemented, in some cases, in hardware, firmware, software, or any combination thereof. The disclosed embodiments may also be implemented as instructions carried by or stored on a transitory or non-transitory machine-readable (e.g., computer-readable) storage medium, which may be read and executed by one or more processors. A machine-readable storage medium may be embodied as any storage device, mechanism, or other physical structure for storing or transmitting information in a form readable by a machine (e.g., a volatile or non-volatile memory, a media disc, or other media device).

In the drawings, some structural or method features may be shown in specific arrangements and/or orderings. However, it should be appreciated that such specific arrangements and/or orderings may not be required. Rather, in some embodiments, such features may be arranged in a different manner and/or order than shown in the illustrative figures. Additionally, the inclusion of a structural or method feature in a particular figure is not meant to imply that such feature is required in all embodiments and, in some embodiments, may not be included or may be combined with other features.

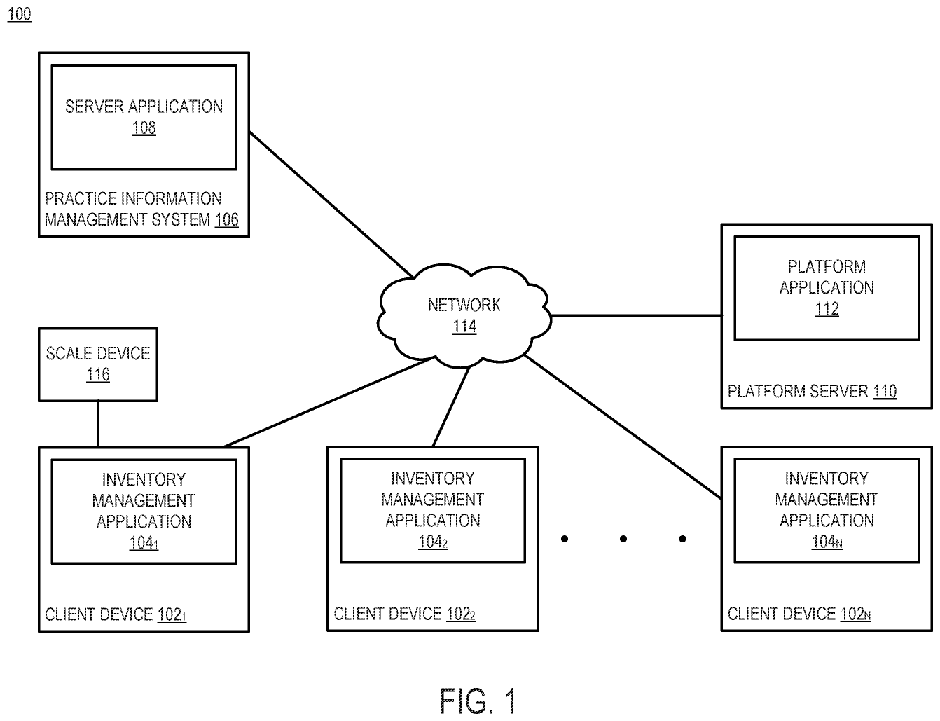

Referring now to FIG. 1, a computing environment 100 in which controlled substance usage data from multiple sources is tracked for generating a DEA-compliant accountability audit report, according to an embodiment. As shown, the computing environment 100 includes client devices 102_{1-N}, a practice information management system (PIMS) 106, and a platform server 110, each interconnected via a network 114 (e.g., the Internet, a local area network, private network, etc.).

The client devices 102_{1-N} may be embodied as a physical computing device (e.g., a desktop computer, laptop computer, mobile device such as a mobile tablet or a smartphone, etc.) or a virtual computing instance executing in a cloud network. A user of the client devices 102_{1-N} may be an individual employed at a medical practice, such as a veterinary practice (e.g., a veterinary clinic, hospital, office with a pharmacy, etc.) that administers treatment to an animal patient, such as dispensing and administering medication to the patient, including controlled substances. As used herein, a controlled substance pertains to a drug or a chemical that is subject to governmental regulations such as the Controlled Substances Act (CSA) with regard to manufacture, possession, and use.

Illustratively, the client devices 102_{1-N} includes an inventory management application 104_{1-N}. The inventory management application 104_{1-N} is a user-side application for a digital log book platform (e.g., hosted on the platform server 110 to be further described herein) and provides a drug inventory management interface enabling the user to record controlled substance usage over the course of the day. The interface may be embodied as a “digital log book” that maintains records of usage and controlled substance inventory of the practice. For example, the user may, via the inventory management application 104_{1-N}, record entries of controlled substance usage into the digital log book provided by the interface. The interface may allow the user to enter, for example, patient information (e.g., name, chart ID, owner name, treatment), drug name, quantity, physician information, approval information, and so on. Further, the inventory management application 104 is integrated with one or more PIMS, such as the PIMS 106 to be further described herein. In an embodiment, the inventory management application 104_{1-N} may be embodied as a mobile “app” or as an application instance running on a web browser executing on the client device 102_{1-N}.

The interconnection of client devices 102_{1-N} over the network 114 and via the platform server 110 enables multiple users to access and update the digital log book contemporaneously, e.g., from different locations within the medical practice or outside the medical practice. For instance, a user at the client device 102₁ may record usage of a controlled substance for a given patient in inventory management application 104₁ while another user at client device 102₂ records usage of a different controlled substance for another patient in inventory management application 104₂. Both updates in usage may propagate over the network 114 to the inventory management applications 104_{1-N}. For example, when the users make the updates, the updates are transmitted to the platform application 112, which in turn processes and transmits the updates to the inventory management applications 104_{1-N}.

The PIMS 106 may be a physical computing system (e.g., a desktop computer, workstation computer, laptop computer, etc.) or a virtual computing instance executing in the cloud. The PIMS 106 can also run on the premises of the veterinary practice or a remote location. As shown, the PIMS 106 further includes a server application 108, providing functionalities of a PIMS, including managing patient data (including maintaining records of controlled substance usage per patient), invoicing, inpatient care workflows, reporting workflows, and so on. In an embodiment, the inventory management applications 104_{1-N} (and platform application 112) populate information managed and input to the PIMS 106 such as patient and client personal identifying information, patient charts and histories, medical practice user data and so on.

In an embodiment, the inventory management applications 104_{1-N} is incorporated into an inventory management platform that includes the platform server 110. In other embodiments, one or a combination of the functions performed by the inventory management applications 104_{1-N} and platform application 112 can be performed by a single application.

In an embodiment, the platform server 110 stores and maintains standardized PIMS data for later processing by the inventory management application 104. To do so, the platform server 110 includes a platform application 112 executing thereon that performs such functions. The platform application 112 may provide an API to the inventory management application 104 to enable communication (e.g., transmission of PIMS data, local controlled usage log data, accountability audit report generation, etc.) between the applications 104 and 112. In other embodiments, the platform application 112 also maintains and stores usage data provided by the client devices 102_{1-N} each executing an instance of the inventory management application 104_{1-N} (e.g., in the event that a client device 102 is a mobile device such as a tablet, and the inventory management application 104 is operated by multiple users). In other further embodiments, the platform server 110 manages data recorded in the digital log book of the inventory management application 104 and tracks inventory and usage of controlled substances during normal operation of the medical practice, such that an audit trail can be ascertained by virtue of the activity being tracked through the platform. The platform application 112 may use APIs provided by the server application 116 to communicate with the PIMS 106.

As stated, controlled substance usage data may be recorded and maintained by the various components of the computing environment 100. For example, the inventory management application 104_{1-N} maintains a controlled substance usage log in a digital log book. Further, the PIMS data includes controlled substance usage information. The platform application 112 (or the inventory management application 104) may validate and reconcile controlled substance usage data of multiple sources. To ensure this accuracy in the controlled substance usage data, the platform application 112 verifies and matches usage data of drugs being tracked from each individual source. The platform application 112 uses identifiable information such as drug name, date of usage, client, or patient chart number, the source from which the usage data was obtained, and other typically recorded data.

In an embodiment, the inventory management applications 104_{1-N} and platform application 112 may manage the inventory balances of controlled substances using virtual container objects. A virtual container object is a data object that represents and corresponds to a physical container used to store up to a predefined quantity of a given controlled substance at the medical practice. In addition to storing information regarding a given controlled substance, the inventory management applications 104_{1-N} and platform application 112 may also associate quantities of a controlled substance to one or more virtual container objects. Because the virtual container objects maintained by the platform correspond on a 1:1 basis to their physical counterparts, managing and tracking the flow of controlled substances via the controller assists in generating a DEA-complaint audit report, as will further be described herein.

In an embodiment, the inventory management applications 104_{1-N} may be integrated with a weighing scale 116 (shown in FIG. 1 as being integrated with the client device 102₁ and inventory management application 104₁). The weighing scale 116 may be embodied as a hardware device configured to measure a weight of an object, such as a physical container of a controlled substance. Once measured, the weighing scale 116 is further configured to transmit the measured weight to the inventory management applications 104_{1-N}. As further described herein, the inventory management application 104_{1-N} may apply the weight measurement in a variety of scenarios. For example, the inventory management application 104_{1-N} may determine a quantity of a controlled substance in a physical container based on a weight and amount per tablet. In so determining, the inventory management application 104_{1-N} is able to identify inaccuracies in manual entries of usage for that controlled substance. The inventory management application 104_{1-N} may also use weight measurements transmitted by the weighing scale 116 to confirm inventory, such as during the closure of a virtual container associated with a physical controlled substance.

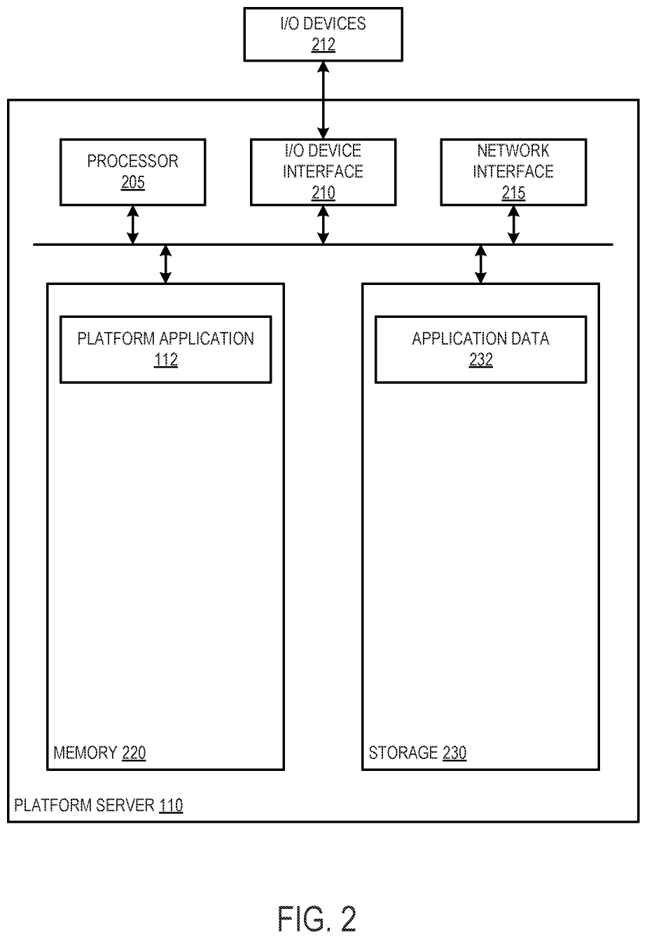

FIG. 2 further illustrates the platform server 110. As shown, platform server 110 includes, without limitation, a central processing unit (CPU) 205, an input/output (I/O) device interface 210, one or more I/O devices 212, a network interface 215, a memory 220, and a storage 230, each interconnected via a hardware bus 217. Of course, the actual platform server 110 will include a variety of additional hardware components not shown. Additionally, in some embodiments, one or more of the illustrative components may be incorporated in, or otherwise form a portion of, another component.

The CPU 205 retrieves and executes programming instructions stored in the memory 220 (e.g., of the platform application 112). The CPU 205 may be embodied as one or more processors, each processor being a type capable of performing the functions described herein. For example, the CPU 205 may be embodied as a single or multi-core processor(s), a microcontroller, or other processor or processing/controlling circuit. In some embodiments, the CPU 205 may be embodied as, include, or be coupled to a field programmable gate array (FPGA), an application-specific integrated circuit (ASIC), reconfigurable hardware or hardware circuitry, or other specialized hardware to facilitate performance of the functions described herein. The hardware bus 217 is used to transmit instructions and data between the CPU 205, storage 230, network interface 215, and the memory 220. CPU 205 is included to be representative of a single CPU, multiple CPUs, a single CPU having multiple processing cores, and the like. The memory 220 may be embodied as any type of volatile (e.g., dynamic random access memory, etc.) or non-volatile memory (e.g., byte addressable memory) or data storage capable of performing the functions described herein. Volatile memory may be a storage medium that requires power to maintain the state of data stored by the medium. Non-limiting examples of volatile memory may include various types of random access memory (RAM), such as DRAM or static random access memory (SRAM). One particular type of DRAM that may be used in a memory module is synchronous dynamic random access memory (SDRAM). In particular embodiments, DRAM of a memory component may comply with a standard promulgated by JEDEC, such as JESD79F for DDR SDRAM, JESD79-2F for DDR2 SDRAM, JESD79-3F for DDR3 SDRAM, JESD79-4A for DDR4 SDRAM, JESD209 for Low Power DDR (LPDDR), JESD209-2 for LPDDR2, JESD209-3 for LPDDR3, and JESD209-4 for LPDDR4. Such standards (and similar standards) may be referred to as DDR-based standards and communication interfaces of the storage devices that implement such standards may be referred to as DDR-based interfaces.

The network interface 215 may be embodied as any hardware, software, or circuitry (e.g., a network interface card) used to connect the platform server 110 over the network 114 and providing the network communication component functions described above. For example, the network interface 215 may be embodied as any communication circuit, device, or collection thereof, capable of enabling communications over the network 114 between the platform server 110 and other devices (e.g., the client device 102, PIMS 106, etc.). The network interface 215 may be configured to use any one or more communication technology (e.g., wired, wireless, and/or cellular communications) and associated protocols (e.g., Ethernet, Bluetooth®, Wi-Fi®, WiMAX, 5G-based protocols, etc.) to effect such communication. For example, to do so, the network interface 215 may include a network interface controller (NIC, not shown), embodied as one or more add-in-boards, daughtercards, controller chips, chipsets, or other devices that may be used by the platform server 110 for network communications with remote devices. For example, the NIC may be embodied as an expansion card coupled to the I/O device interface 210 over an expansion bus such as PCI Express.

The I/O device interface 210 allows the I/O devices 212 to communicate with hardware and software components of the platform server 110. For example, the I/O device interface 210 may be embodied as, or otherwise include, memory controller hubs, input/output control hubs, integrated sensor hubs, firmware devices, communication links (e.g., point-to-point links, bus links, wires, cables, light guides, printed circuit board traces, etc.), and/or other components and subsystems to facilitate the input/output operations. In some embodiments, the I/O device interface 210 may form a portion of a system-on-a-chip (SoC) and be incorporated, along with one or more of the CPU 205, the memory 220, and other components of the platform server 110.

The I/O devices 212 may be embodied as any type of I/O device connected with or provided as a component to the platform server 110. I/O devices such as keyboards, mice, and printers may be included as I/O devices 212 (e.g., to enter controlled substance usage information once administered to a patient, print DEA accountability reports, etc.). Illustratively, the memory 220 includes the platform application 112.

The storage 230 may be embodied as any type of devices configured for short-term or long-term storage of data such as, for example, memory devices and circuits, memory cards, hard disk drives (HDDs), solid-state drives (SSDs), or other data storage devices. The storage 230 may include a system partition that stores data and firmware code for the storage 230. The storage 230 may also include an operating system partition that stores data files and executables for an operating system. Illustratively, the storage 230 includes application data 232, which may be indicative of controlled substance data stored and managed by the platform. For instance, the application data 232 can include data of one or more medical practices, such as inventory management application user data, digital log books, invoices, and data that may otherwise be locally stored on a client device 102_{1-N} executing the inventory management application 1-N or the PIMS 106.

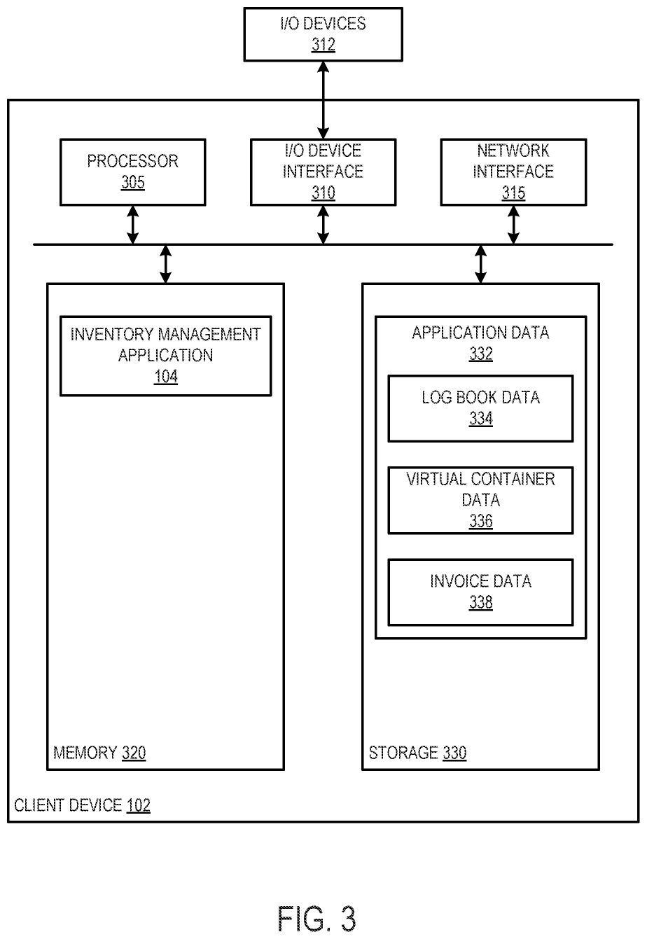

FIG. 3 further illustrates an example client device 102, which may be representative of any of the client devices 102_{1-N}. As shown, the client device 102 includes, without limitation, a CPU 305, an I/O device interface 310, one or more I/O devices 312, and a network interface 315, memory 320, and storage 330, each interconnected via a hardware bus 317. Of course, the actual client device 102 will include a variety of additional hardware components not shown. Additionally, in some embodiments, one or more of the illustrative components may be incorporated in, or otherwise form a portion of, another component. The CPU 305, I/O device interface 310, I/O devices 312, network interface 315, hardware bus 317, memory 320, and storage 330 are similar to the respective CPU 205, I/O device interface 210, I/O devices 212, network interface 215, hardware bus 217, memory 220, and storage 230 of the platform server 110. Illustratively, the memory 320 includes the inventory management application 104, which may represent any of the inventory management applications 104_{1-N}.

The storage 332 includes application data 332. As shown, the application data 332 may include log book data 334, virtual container data 336, and invoice data 338. The log book data 334 may be a local instance of the digital log book provided by the platform application 112 that a user can view and update. The virtual container data 336 may be local instances of virtual containers each representing a physical container inventory of controlled substances (and/or other substances managed by the medical practice) as well as providing mappings between a given virtual container and physical container, to be described further herein. The invoice data 338 may be embodied as local instances of financial invoices issued by or to the medical practice associated with patient examinations, controlled substance intake, controlled substance dispensing, and the like. In some embodiments, the application data 332 may also include additional controlled substance log data and audit reports generated by the platform application 112 (or inventory management application 104).

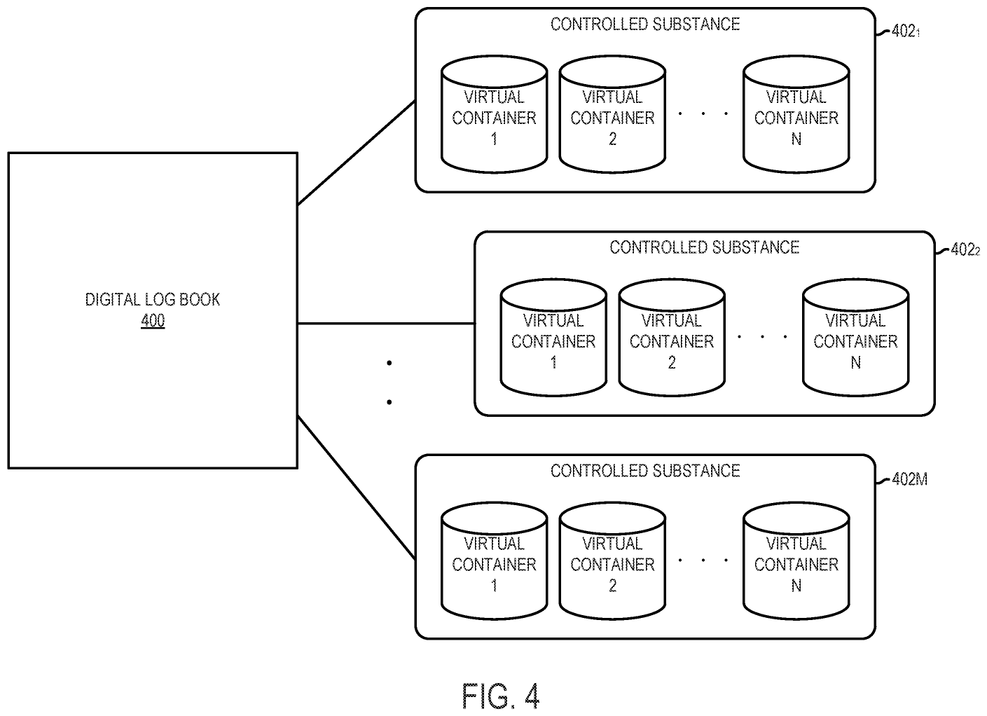

FIG. 4 is a conceptual diagram depicting an abstraction an example digital log book 400 managed by a medical practice via an inventory management application 104_{1-N}. In this example, the digital log book 400 may include a number of entries (also referred herein as “logs”) corresponding to various inventory events, such as intake of a controlled substance to facility of the medical practice, a transfer of a controlled substance from a given facility to another facility of the medical practice, dispensing or administration of a controlled substance to a patient, the opening of a physical container of a controlled substance, closing of a physical container of a controlled substance, disposal or wastage of a controlled substance, and so on.

As a user enters transactions or entries that reference controlled substances in the PIMS 106 or the inventory management application 104_{1-N}. the inventory management application 104_{1-N} (or platform application 112) may associate the controlled substance with the digital log book 400. Illustratively, controlled substances 402_{1-M} are associated with the digital log book 400. Further, as shown, quantities each of the controlled substances 402_{1-M} may be apportioned into one or more virtual containers 1-N. As stated, a virtual container 1-N is a data object representing and corresponding to a real-world physical container of a given controlled substance 402_{1-M} inventoried by the medical practice. Each of the virtual containers 1-N may include attributes associated with an inventories physical container, such as name, initial quantity, current quantity, personnel associated with inventory events (e.g., intaking the physical container, an event history associated with the virtual container), location of the physical container, status of whether the physical container is open or closed, and the like.

Advantageously, the inventory management application 104 may evaluate virtual containers to ensure a proper accounting of controlled substances managed at the medical practice. For example, the inventory management application 104 may identify events linked to virtual containers 1-N not currently present in the digital log book 400 and populate the digital log book 400 with entries corresponding to the missing data.

As another example, in some embodiments, the inventory management application 104 may, based on the virtual containers present in the application and the attributes associated therewith, generate reports and other types of documentation or data. For instance, the inventory management application 104 may create or recreate a digital log book entries based on virtual container data managed within the inventory management application 104. Such an example may occur when transactions listed in invoices do not have corresponding entries in a digital log book, such as the digital log book 400. Assume that a medical practice staff user creates an invoice in the PIMS 106 for controlled substances given to a patient, in which the invoice can include details of the controlled substance and a quantity (e.g., buprenorphine of a quantity of 0.2 mL), date, patient, container identification information, and client (if applicable). The inventory management application 104 may periodically query the PIMS 106 for updates and retrieve invoices for the controlled substance. The inventory management application 104 may detect that the information provided in the invoices do not match. Once detected, the inventory management application 104 may flag the invoice for further attention by a user. In an embodiment, the inventory management application 104 may provide a list of such invoices, such as through a graphical user interface (GUI) listing invoices that do not have corresponding digital log book entries. The user may then select one of the listed invoices and request to generate a controlled substance log entry for the digital log book. The inventory management application 104 may then identify (e.g., based on the information listed in the invoice) the virtual container used in the underlying transaction and match usage data for the virtual container (e.g., identify an amount drawn) with the invoice information.

In an embodiment, the inventory management application 104 may also generate entries from inventory usage data provided by the PIMS 106 that are not present in a digital log book (e.g., the digital log book 400). The inventory management application 104 may periodically query the PIMS 106 for updates and retrieve inventory usage data for the controlled substance. The inventory management application 104 may detect that the information provided in the PIMS 106 inventory usage data does not match. Once detected, the inventory management application 104 may flag the inventory usage data for further attention by a user. In an embodiment, the inventory management application 104 may provide a list of such inventory usage entries, such as through a graphical user interface (GUI) listing invoices that do not have corresponding digital log book entries. The user may then select one of the listed invoices and request to generate a controlled substance log entry for the digital log book. The inventory management application 104 may then identify (e.g., based on the information provided in the inventory usage data) the virtual container used in the underlying transaction and match usage data for the virtual container (e.g., identify an amount drawn) with the inventory usage information.

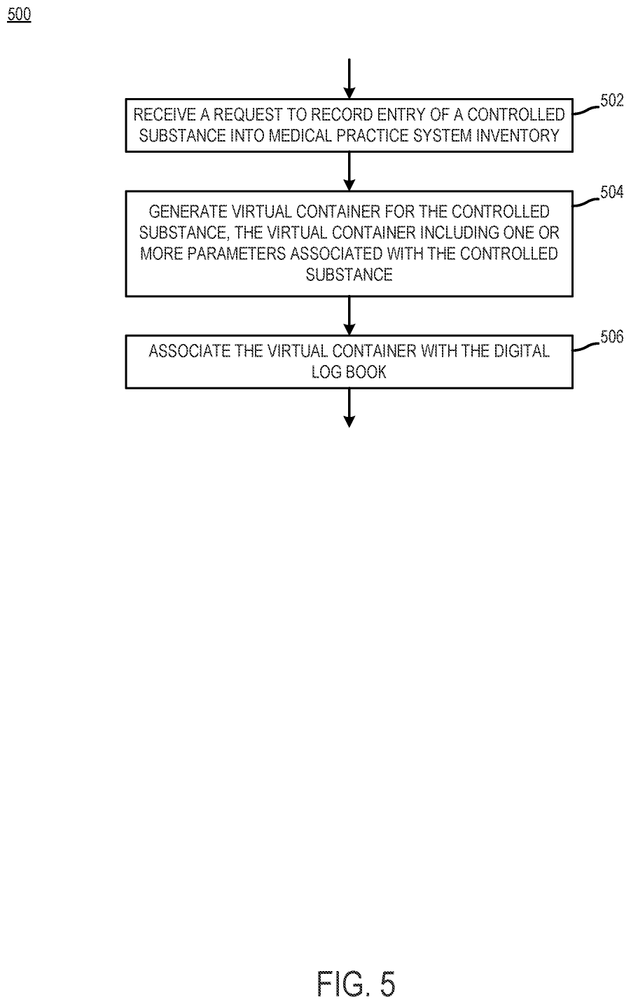

FIG. 5 illustrates a method 500 for linking a controlled substance to a digital log book using a virtual container, according to an embodiment. For example, the inventory management application 104 (or platform application 112) may perform method 500 during intake of a physical container of a controlled substance into the systems of a medical practice. As another example, the inventory management application 104 (or platform application 112) may perform method 500 after the controlled substance has been entered into the systems of the medical practice (e.g., during a routine inventory check of substances at a facility of the medical practice, during integration or migration of legacy system data, etc.).

As shown, the method begins in block 502, in which the inventory management application 104 (or platform application 112) receives a request to record entry of a controlled substance into a system inventory of a medical practice. The request may include attributes associated with the controlled substance, such as a drug name, a quantity, expiration date, facility name, medical practice name, identifying information associated with an underlying physical container, etc. Further, the request may be manually submitted (e.g., via the inventory management application 104) or automatically sent from the PIMS 106.

In an embodiment, the inventory management application 104 may include weight measurement data provided by the scale device 116 for improved accuracy and tracking. For example, in making the request through a user face of the inventory management application 104, a user may place the controlled substance on the scale device 116 and taking an initial weight measurement of the controlled substance in various configurations (e.g., the weight of the container as provided, the weight of the open container). The user (or PIMS 106) may also provide a quantity and dosage per pill of the controlled substance provided by a manufacturer or supplier. Doing so allows the inventory management application 104 to correlate the weight of the container with the quantity of the substance for more accurate tracking as controlled substances are removed from the container over the course of operation of the medical practice.

In block 504, the inventory management application 104 (or platform application 112) generates a virtual container for the controlled substance based on the request. The inventory management application 104 may assign various attributes to the virtual container, such as the attributes provided by the request as well as status information (whether the virtual container should be considered “open” or “closed” for the purposes of dispensing and tracking), a maximum capacity, inventory balance, and a unique identifier for the virtual container. In an embodiment, the inventory management application 104 may associate the virtual container with a physical container, e.g., by assigning an attribute associated with the physical container to the virtual container (e.g., serial numbers or other identifiers associated with the physical container). During this step, the inventory management application 104 may also perform additional inconsistency detection and reconciliation measures, such as identifying whether the container has previously been input into any of the medical practice systems (e.g., the PIMS 106, platform application 112, inventory management application 104, etc.), identifying whether another user has entered the same controlled substance into another instance of the inventory management application 104, whether the controlled substance was entered incorrectly in another system (e.g., the PIMS 106), etc. If any inconsistencies have been identified, the inventory management application 104 (or platform application 112) may flag the entry for additional review by the user or automatically perform reconciliation based on predefined rules.

In block 506, the inventory management application 104 associates the virtual container with the digital log book of the medical practice. For example, to do so, the inventory management application 104 (or platform application 112) may assign an attribute to the virtual container that includes an identifier associated with the digital log book. The inventory management application 104 may also record the identifier for the virtual container in the digital log book. In addition, for instances in which the control substance is being entered during intake at the medical practice, the inventory management application 104 may generate a digital log book entry for the intake and include the attributes associated with the virtual container including the unique identifier to the entry.

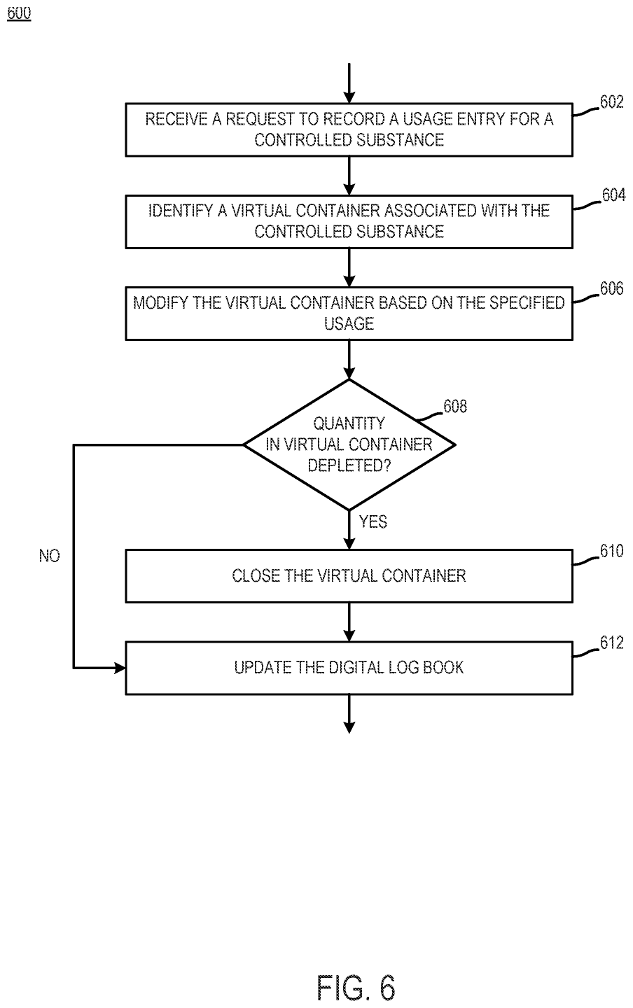

FIG. 6 illustrates a method 600 for managing virtual container data associated with a controlled substance, according to an embodiment. The inventory management application 104 may perform the method 600 during regular operation of the medical practice as controlled substances are administered, dispensed, or otherwise used throughout the day. As shown, the method 600 begins in block 602, in which the inventory management application 104 (or platform application 112) receives a request to record a usage entry for a controlled substance. The request may include parameters associated with the usage, such as the type of usage, date and time of the usage, quantity amount used, patient associated with the usage, client associated with the usage, medical personnel associated with the usage, attributes associated with the underlying physical container, and any other data that is relevant for compliance reporting purposes.

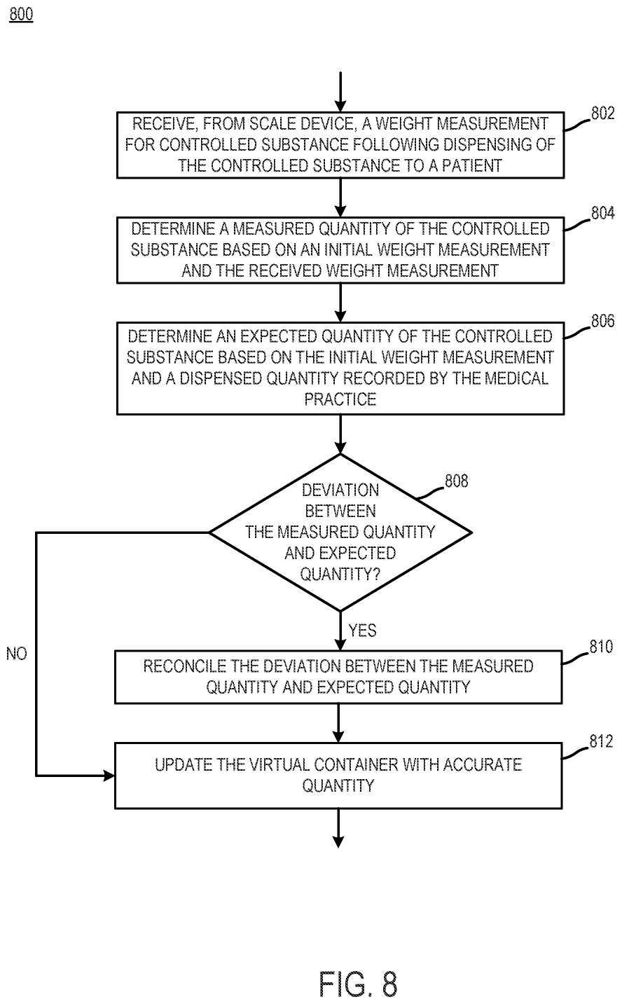

In block 604, the inventory management application 104 (or platform application 112) identifies a virtual container associated with the controlled substance based on the request. For example, to do so, the inventory management application 104 may evaluate the physical container identifier provided in the request against existing virtual containers to identify a matching virtual container. In block 606, the inventory management application 104 (or platform application 112) updates the virtual container based on the specified usage. For example, the inventory management application 104 may deduct the specified usage quantity from the current quantity. In an embodiment, the inventory management application 104 may use the scale device 116 to determine the remaining quantity amount and also reconcile inconsistencies in the parameters provided in the original request. FIG. 8 depicts such a method in further detail.

In block 608, the inventory management application 104 (or platform application 112) determines whether the quantity of the virtual container has been depleted, e.g., if the corresponding value is 0 (or at an amount less than 1). If so, then in block 610, the inventory management application 104 (or platform application 112) may close the virtual container. To do so, the inventory management application 104 may update a status attribute of the virtual container as closed, indicating that the virtual container (and underlying physical container) is empty for tracking and reporting purposes. By setting the virtual container as finished, the inventory management application 104 (and the platform application 112) will no longer record logs against the virtual container for the controlled substance. If the virtual container is not yet depleted (or following the closing of the virtual container), the method 600 goes to block 612, in which the inventory management application 104 updates the digital log book. More particularly, the inventory management application 104 generates a usage entry corresponding to the request for the digital log book and performs any reconciliation measures previously described herein.

In an embodiment, the platform application 112 may manage container logs for the controlled substance. More particularly, the recorded usage associated with a given controlled substance may be logged along with the container. For example, assume that the veterinary practice administers Buprenorphine to a dog patient. In such a case, the platform application 112 may track, through log data sent by a user (or automatically retrieved) from the inventory management application 104, an quantity of the substance administered and a container ID from which the substance was taken.

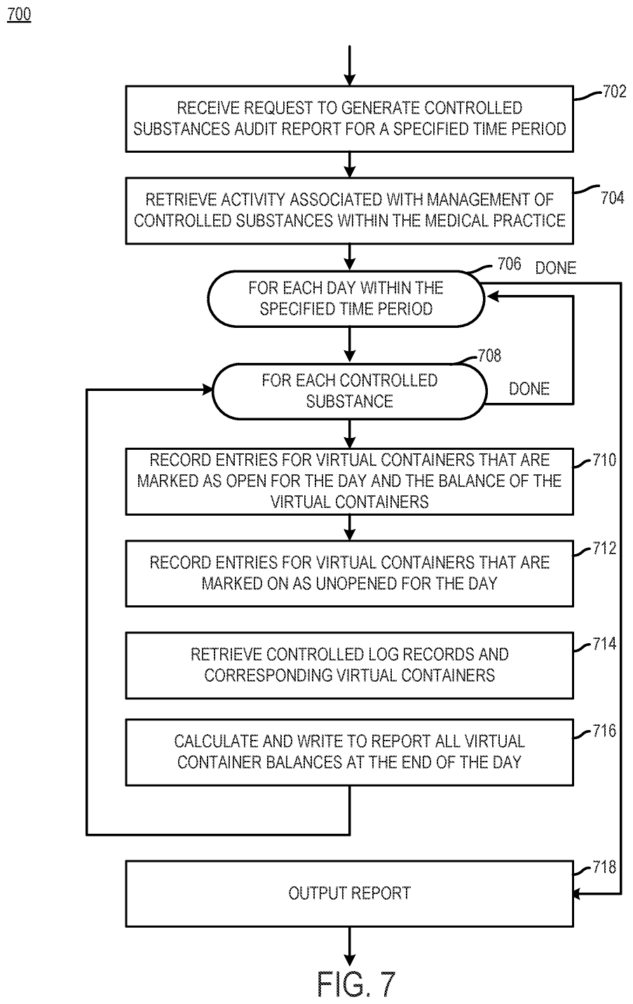

FIG. 7 is a method 700 for generating an accountability audit report of controlled substance inventory and usage for a medical practice, according to an embodiment. As shown, the method 700 begins in block 702, in which the platform application 112 receives a request to generate an accountability audit report for a specified time period. For instance, a user may, via the inventory management application 104 on the client device, click on a button within a user interface indicating to generate the report. In block 704, the platform application 112 retrieves, from a data store (e.g., the storage 230 or some other location such as on a cloud storage server), activity associated with the management of controlled substances within the medical facility. To do so, the platform application 112 may instantiate a job to retrieve from the data store all relevant data, e.g., activity data associated with the specified time period.

In blocks 706 and 708, the method 700 iterates the following steps for each day of the specified time period and for each controlled substance for that day. In block 710, the platform application 112 records entries for virtual containers that are marked as open for the day. The platform application 112 also records the balance of the associated virtual containers. In block 712, the platform application 112 records entries for virtual containers that are marked as unopened for the day. The platform application 112 writes, to the report, all drug usage for the date, which can include regular logs (e.g., N quantity given to a patient X), accidental waste, hub loss, and the like.

In block 714, the platform application retrieves controlled log records and corresponding virtual containers listed in the records. In block 716, the platform application 112 calculates and writes to the resulting report all virtual container balances for the controlled substance corresponding to the end of the day. By retrieving all controlled log records as well as virtual containers, the platform application 112 is able to evaluate relevant totals (e.g., start and end balances) and also allows evaluation of each draw from the virtual container to allow an individual user to see how the end balance was reached. If there are no inconsistencies, the start of day balance subtracted from the usage should match the end of day balance for that controlled substance. Once the controlled substances have been iterated through for that day, the method iterates to the next day and performs the aforementioned steps described for blocks 710 through 716. In block 718, the platform application 112 may output the report to the inventory management application 104. From there, the inventory management application 104 may display the report on the client device 102. The user may then print the report via the inventory management application 104.

Referring now to FIG. 8, a method 800 for reconciling inconsistent quantity reporting for a digital log book is now shown. The method 800 begins in block 802, in which the inventory management application 104 receives, from a scale device (e.g., scale device 116), a weight measurement for a controlled substance following a usage of the controlled substance, such as the dispensing of the controlled substance to a patient. For instance, after a medical professional user has administered the controlled substance, the user may weigh the physical container on the scale device 116, which in turn transmits the measured weight to the inventory management application 104 (and platform application 112).

In block 804, the inventory management application 104 determines a measured quantity of the controlled substance based on an initial weight measurement (e.g., taken at intake of the underlying physical container of the controlled substance) and the received weight measurement. In block 806, the inventory management application 104 determines an expected quantity of the controlled substance. The inventory management application 104 may do so based on the initial weight measurement and a dispensed quantity recorded by the medical practice.

In block 808, the inventory management application 104 determines whether a deviation between the measured quantity and the expected quantity exists. If so, then the inventory management application 104 reconciles the deviation. For example, the inventory management application 104 may flag the deviation in the graphical user interface for a user to review and provide input. As another example, the inventory management application 104 may automatically reconcile the deviation based on a set of predefined rules. If no deviation exists (or after the deviation is reconciled), then the method 800 proceeds to block 812, in which the inventory management application 104 updates the virtual container with the accurate quantity.

While the foregoing is directed to embodiments of the present disclosure, other and further embodiments of the disclosure may be devised without departing from the basic scope thereof, and the scope thereof may be determined by the example claims that follow.

What is claimed is:

1. A system comprising:
one or more processors;
a memory storing a plurality of instructions, which, when executed on the one or more processors, causes the system to:
provide a platform application enabling, from a plurality of computing devices, entry of inventory and usage records of one or more controlled substances for a medical practice;
receive, via the platform application from a first computing device, a request to record entry of a first controlled substance into a digital log book associated with the medical practice, the request specifying at least a date of the entry and a quantity of the first controlled substance;
generate, via the platform application, a virtual container to associate with the first controlled substance, the virtual container including the specified information; and
associate, via the platform application, the virtual container to the digital log book.

2. The system of claim 1, further comprising a scale configured to obtain weight measurements of the one or more controlled substances and transmit signals indicative of the weight measurements, wherein the plurality of instructions further causes the system to:
receive, from the scale, an initial weight measurement for the first controlled substance;
update the virtual container associated with the first controlled substance to include the initial weight measurement of the first controlled substance;
receive an indication of a dispensing of the first controlled substance to a patient, the indication including a dispensed quantity of the first controlled substance recorded by the first computing device;
receive, from the scale, a second weight measurement for the first controlled substance following the dispensing of the first controlled substance;
identify a measured quantity based on the initial weight measurement and the second weight measurement of the first controlled substance.

3. The system of claim 2, wherein the plurality of instructions further causes the system to:
determine an expected quantity of the first controlled substance based on the initial weight measurement and the dispensed quantity recorded by the first computing device; and
detect, based on the measured quantity, a deviation from the expected quantity.

4. The system of claim 3, wherein the plurality of instructions further causes the system to:
reconcile the deviation between the expected quantity and measured quantity; and
update the virtual container associated with the first controlled substance based on the reconciliation.

5. The system of claim 4, wherein to reconcile the deviation comprises to update, based on the deviation, a current quantity associated with the first controlled substance according to a set of predefined rules.

6. The system of claim 4, wherein the plurality of instructions further causes the system to:
detect that the quantity associated with the virtual container has depleted; and
mark the virtual container as closed in the digital log book.

7. The system of claim 1, wherein the plurality of instructions further causes the system to:
receive, via the platform application and from the first computing device, a request to generate an audit report for the inventory and usage of the one or more controlled substances over a specified time period;
determine an accounting of the controlled substances for the specified time period based on at least a distribution of the controlled substances to respective container objects over the specified time period; and
generate the audit report based on the accounting of the controlled substances.

8. The system of claim 1, wherein the virtual container includes one or more parameters, the one or more parameters including at least one of the quantity of the first controlled substance, a current inventory of the first controlled substance, a capacity of the container object, and an indication of whether the container object is open, unopened, or closed.

9. The system of claim 1, wherein the plurality of instructions further causes the system to:
receive, via the platform application, one or more updates relating to a usage of the first controlled substance from a practice management system of the medical practice; and
update, via the platform application, the digital log book based on the one or more updates.

10. The system of claim 9, wherein the plurality of instructions further causes the system to:
update one or more attributes of the virtual container based on the one or more updates.

11. A method comprising:
providing a platform application enabling, from a plurality of computing devices, entry of inventory and usage records of one or more controlled substances for a medical practice;
receiving, via the platform application from a first computing device, a request to record entry of a first controlled substance into a digital log book associated with the medical practice, the request specifying at least a date of the entry and a quantity of the first controlled substance;
generating, via the platform application, a virtual container to associate with the first controlled substance, the virtual container including the specified information; and
associating, via the platform application, the virtual container to the digital log book.

12. The method of claim 11, further comprising:
receiving, from a scale configured to obtain weight measurements of the one or more controlled substances and transmit signals indicative of the weight measurements, an initial weight measurement for the first controlled substance;
updating the virtual container associated with the first controlled substance to include the initial weight measurement of the first controlled substance;
receiving an indication of a dispensing of the first controlled substance to a patient, the indication including a dispensed quantity of the first controlled substance recorded by the first computing device;
receiving, from the scale, a second weight measurement for the first controlled substance following the dispensing of the first controlled substance;
identifying a measured quantity based on the initial weight measurement and the second weight measurement of the first controlled substance.

13. The method of claim 12, further comprising:
determining an expected quantity of the first controlled substance based on the initial weight measurement and the dispensed quantity recorded by the first computing device; and
detecting, based on the measured quantity, a deviation from the expected quantity.

14. The method of claim 13, further comprising:
reconciling the deviation between the expected quantity and measured quantity; and
updating the virtual container associated with the first controlled substance based on the reconciliation.

15. The method of claim 14, wherein reconciling the deviation comprises updating, based on the deviation, a current quantity associated with the first controlled substance according to a set of predefined rules.

16. The method of claim 14, further comprising:
detecting that the quantity associated with the virtual container has depleted; and
changing a status attribute of the virtual container as closed in the digital log book.

17. The method of claim 11, further comprising:
receiving, via the platform application and from the first computing device, a request to generate an audit report for the inventory and usage of the one or more controlled substances over a specified time period;
determining an accounting of the controlled substances for the specified time period based on at least a distribution of the controlled substances to respective container objects over the specified time period; and
generating the audit report based on the accounting of the controlled substances.

18. The method of claim 11, wherein the virtual container includes one or more parameters, the one or more parameters including at least one of the quantity of the first controlled substance, a current inventory of the first controlled substance, a capacity of the container object, and an indication of whether the container object is open, unopened, or closed.

19. The method of claim 11, further comprising:
receiving, via the platform application, one or more updates relating to a usage of the first controlled substance from a practice management system of the medical practice;
updating, via the platform application, the digital log book based on the one or more updates.

20. The method of claim 19, further comprising:
updating one or more attributes of the virtual container based on the one or more updates.
